(19) United States
(12) Patent Application Publication
(10) Pub. No.: US 2015/0346181 A1
(43) Pub. Date: Dec. 3, 2015

(54) DEVICE AND SYSTEM TO DETERMINE ENGINE LUBRICATION HEALTH

(71) Applicant: Caterpillar Inc., Peoria, IL (US)

(72) Inventors: Walter S. Isenberg, Frankfort, IN (US); Kevin P. Storms, Darlington, IN (US)

(73) Assignee: Caterpillar Inc., Peoria, IL (US)

(21) Appl. No.: 14/826,262

(22) Filed: Aug. 14, 2015

(51) Int. Cl. G01N 33/28

(52) U.S. Cl. CPC G01N 33/2888

(57) ABSTRACT

A health analysis system for an engine lubrication system is disclosed. The engine lubrication system includes a sump, a lubricant quality sensor to detect quality related to at least one parameter of a lubricant, and a conduit. The health analysis system includes a bypass line connected from the conduit to the sump to facilitate passage of a sample engine lubricant. Further, a patch containment assembly is included that has a first housing with a first passage. A second housing, defining a second passage, is removably connectable to the first housing. Moreover, a patch member is removably positioned within one of the first housing or the second housing. The first passage is in fluid communication with the second passage through the patch member.

### TECHNICAL FIELD

The present disclosure relates generally to an apparatus and a method to analyze health of a lubricant in lubrication systems of pressure-lubricated components. More specifically, the present disclosure relates to a modular unit, which is connectable externally to units, such as an engine or other components, and infers the quality of the lubricant based on a lubricant sample collected.

### BACKGROUND

Engines applied in construction machinery, for example, have rotating and moving components such as gears and bearing systems. Such components are typically provided with sufficient lubrication to reduce friction and provide cooling, as it is desirable for such components to operate with minimal attention, for prolonged periods. However, a certain percentage of such components and other engine parts remain prone to failures. Some of these failures introduce contamination in an associated lubricant, causing a degradation of lubricant health.

Conventional diagnostic tests applied in lubrication systems that rely on methods such as Scheduled Oil Sampling (SOS) may be limited in the detection of a rapidly advancing component failure or a sudden ingestion of debris or contaminants in the lubricant. Furthermore, in many situations, a change of lubricant is proposed based upon a measured use of the engine that employs the lubricant, rather than the actual condition of the lubricant. Therefore, it would be desirable to monitor the condition of the lubricant so that data may be obtained about deterioration or contamination in the lubricant before the occurrence of a failure of the engine or the engine components.

U.S. Pat. Nos. 4,176,545 A relates to an apparatus and an electrical system for sensing wear within an engine. Although the '545 reference discusses the provision for detecting particles in a fluid stream, such as a lubricant, no solution is provided to utilize the apparatus in multiple lubrication systems. Moreover, room remains to further simplify a health monitoring system, which may be applied in the absence of external power, such as electrical power.

Accordingly, the system and method of the present disclosure solves one or more problems set forth above and other problems in the art.

### SUMMARY OF THE INVENTION

Various aspects of the present disclosure illustrate a health analysis system for an engine lubrication system. The engine lubrication system includes a sump, a lubricant quality sensor to detect quality pertaining at least one parameter of a lubricant, and a conduit. The health analysis system includes a bypass line connected from the conduit to the sump to facilitate passage of a sample engine lubricant. Further, a patch containment assembly is included that has a first housing with a first passage. A second housing with a second passage is removably connectable to the first housing. Furthermore, a patch member is removably disposed within one of the first housing or the second housing, or both. The first passage is in fluid communication with the second passage through the patch member.

### BRIEF DESCRIPTION OF THE DRAWINGS

FIG. 1 illustrates an isometric view of an exemplary engine, in accordance with the concepts of the present disclosure;

FIG. 2 is a partial side view of the engine of FIG. 1, illustrated in conjunction with a health analysis system, in accordance with the concepts of the present disclosure;

FIG. 3 is an enlarged view of a patch containment assembly of the health analysis system of FIG. 2, which illustrates a partial cross-section of the patch containment assembly, in accordance with the concepts of the present disclosure; and

FIG. 4 is a flowchart of an exemplary method of operation of the health analysis system, in accordance with the concepts of the present disclosure.

### DETAILED DESCRIPTION

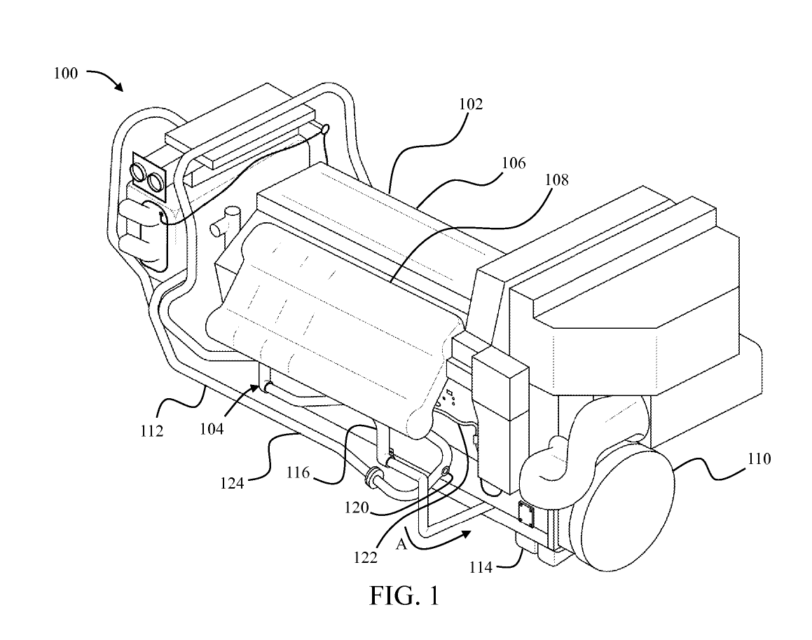

Referring to FIG. 1, there is shown an exemplary power system 100. The power system 100 may be an engine 102, such as a spark-ignition engine or a compression ignition engine, which may be applied in construction machines, such as track-type tractors, hydraulic excavators, wheel loaders, motor graders, and large mining trucks. The engine 102 may be one among the many internal combustion systems. For example, the engine 102 may be fueled by gasoline, diesel, biodiesel, natural gas, propane, hydrogen, combinations thereof, or any other combustion fuel known in the art. The engine 102 may embody a V-type, hemi-type, inline, or any other known configuration.

Nonetheless, it will be appreciated that aspects of the present disclosure are directed to a lubrication system 104 of the engine 102. It may be well suited for one to apply and extend an applicability of the present disclosure to other units that employ lubrication systems. For example, lubrication systems in generator sets, transmission units, work implements, fuel systems, drivetrains, and the like, may suitably benefit from one or more aspects of the present disclosure. Concepts of the present disclosure are also applicable to lubrication systems associated with various other pressure-lubricated components and systems. An extension of the application to domestic and commercial application may also be contemplated.

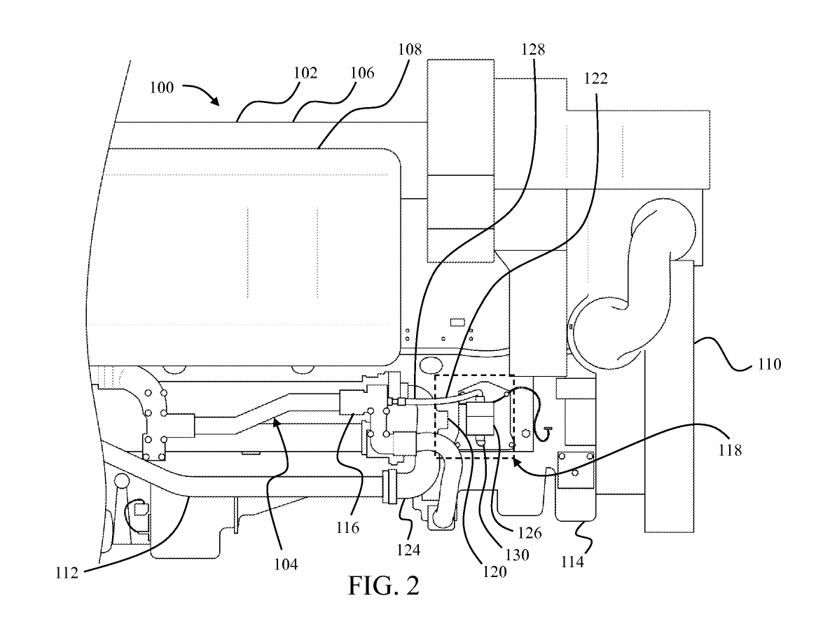

Referring to FIGS. 1 and 2, the engine 102 includes an engine block 106, and a number of components and sub-systems, such as a cylinder head 108 and a flywheel 110, as shown. Collectively, the cylinder head 108 and the flywheel 110 may be referred to as sub-components 108 and 110. The engine 102 is also inclusive of the lubrication system 104 that supplies lubricant to multiple components associated with the sub-components 108 and 110. It will be appreciated that the lubrication system 104 supplies lubricant to other sub-component, units and sub-systems, within the engine 102, such as a camshaft bearing, a crankshaft bearing, rocker arm assemblies, pistons, counter drives, and the like, as well, but which are not shown for ease in clarity and understanding. Therefore, discussions pertaining to the sub-components 108 and 110 may be suitably applied to other sub-systems and units of the engine 102 as well, and the representation of the sub-components 108 and 110 need to be viewed as being purely exemplary in nature.

Generally, the engine block 106 includes an oil gallery (not shown) through which a lubricant is pumped and supplied to each sub-system, such as the sub-components 108 and 110, within the engine 102. As lubrication is provided to multiple portions of the engine 102, a number of channels and lines are structured and routed in and around the engine 102 that facilitate a supply of the lubricant. Cumulatively, these channels and lines are represented by a lubrication line 112. For ease of depiction and understanding, however, the lubrication line 112 is shown only as an individual lubricant transfer conduit. The lubrication line 112 may represent a portion that extends from the sub-systems of the engine 102 to be routed for a return path of a used lubricant into a sump 114. This may occur in a direction, A, as shown in FIG. 1. This flow of lubricant is executed through a used lubricant conduit 116 of the lubrication line 112. The used lubricant conduit 116 may be referred to as a conduit 116, for ease of reference.

Referring to FIG. 2, a health analysis system 118, incorporated with the conduit 116 and associated with the lubrication line 112, is shown. The health analysis system 118 is adapted to analyze a quality of the lubricant flowing through the bypass line 122 and health of the lubrication system 104. The health analysis system 118 includes a lubricant quality sensor 120, a bypass line 122, and a patch containment assembly 126. According to the aspects of the present disclosure, a quality of the lubricant and a health of the lubrication system 104 is deduced from a visual inspection of the remains captured in the patch containment assembly 126 and an output gathered from the lubricant quality sensor 120.

The lubricant quality sensor 120 may be dedicated to the monitoring of at least one parameter pertaining to the quality of the lubricant. The lubricant quality sensor 120 is connected to an auxiliary lubrication line 124. However, it is contemplated that lubricant quality sensor 120 may be located elsewhere within the lubrication system 104. For example, lubricant quality sensor 120 may be located within a lubricant pan (not shown) of the engine 102. In an embodiment, the lubricant quality sensor 120 may be configured to sense a condition of conductivity of the lubricant, and to generate a signal indicative of the conductivity. The generated signal may be communicated to an ECM. However, such conditions may be associated with other quality parameters of the lubricant as well. For instance, the condition could be an amount of soot entrained within the lubricant, a temperature of the lubricant, a viscosity of the lubricant, pH factor of the lubricant, alkali level in the lubricant, total acid or base number of the lubricant, or another known conditions. In an embodiment, the lubricant quality sensor 120 is inclusive of at least one of an analog or a digital reader. In some embodiments, the lubricant quality sensor 120 is located on the lubrication line 112, so as to provide an operator with an outward reference point that indicates a quantity condition of the lubricant, based on the detection of the at least one quality parameter of the lubricant.

The bypass line 122 is connected between the lubricant return conduit 116 and the sump 114. The bypass line 122 may generally be an aftermarket fitment, which may be selectively removable from the conduit 116 and the sump 114. For example, the bypass line 122 is a tube, pipe, or a hose, bent into a form so as to route the used lubricant from the conduit 116 to the sump 114. In the absence of the bypass line 122, a lubricant may flow through the conduit 116 and be returned into the sump 114. However, if the bypass line 122 is connected to the lubrication system 104, a portion of a used lubricant passes through the bypass line 122 as well, into the sump 114.

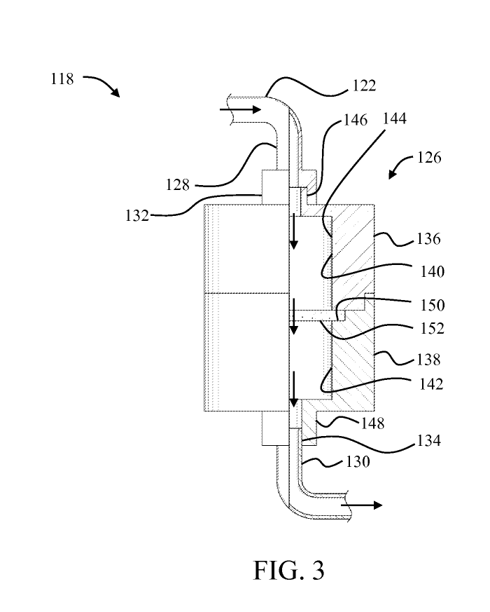

Referring to FIGS. 2 and 3, the bypass line 122 is generally divided into two separate tube portions—namely, an inlet tube portion 128 and an outlet tube portion 130. The inlet tube portion 128 extends from the conduit 116, while the outlet tube portion 130 is connected to the sump 114. The inlet tube portion 128 includes a first connector 132 (FIG. 3), while the outlet tube portion 130 includes a second connector 134 (FIG. 3). In that manner, the tube portions 128 and 130 are selectively connectable to each other by a connection between the first connector 132 and the second connector 134. As shown, the first connector 132 is a female connector, while the second connector 134 is a counter-mating, male connecter. Upon a connection between the first connector 132 and the second connector 134, the inlet tube portion 128 is fluidly connected with the outlet tube portion 130, thus forming the bypass line 122, as shown in FIGS. 1 and 2. A connection between the first connector 132 and the second connector 134 may be facilitated by tight-fit or press-fit connection method. This configuration allows a used sample lubricant to pass through the bypass line 122 from the conduit 116 to the sump 114, during a normal operation of the lubrication cycle. In general, connectors 132 and 134 may be widely available standardized connector units that is adapted for relatively quick connections and disconnections from associated connection points.

Referring to FIG. 3, details pertaining to the patch containment assembly 126 is laid out. The patch containment assembly 126 is a modular unit that facilitates determination of a quality of the lubricant. Such a determination is carried out by ordinally and visually inspecting a measure of the contaminants within the sample lubricant flow through the bypass line 122. The patch containment assembly 126 may be examined by an operator at regular intervals, for example. As shown in the embodiment, the patch containment assembly 126 is fluidly connected with the bypass line 122 and facilitates a portion of the lubricant flowing through the bypass line 122 to flow through the patch containment assembly 126 as well. In so doing, at least a sample of the lubricant flowing through the bypass line 122 is analyzed by a selective inspection strategy of the patch containment assembly 126, and, therefore, a quality of the lubricant and health of the lubrication system 104 is monitored.

In further detail, the patch containment assembly 126 includes a first housing 136 and second housing 138. Both the housings 136 and 138 are substantially cylindrical in structure, although the patch containment assembly 126 may embody other configurations, such as a polygonal configuration. The first housing 136 is removably and co-axially attached to the second housing 138. Such an attachment may be threadably arranged, for example. However, a snap fit engagement between the first housing 136 and the second housing 138 may also be contemplated, or a fitting that is similar to a luer-lock fitting is also possible. In an embodiment, a suction fitting is also envisioned, which may restrict leakage of the lubricant from the patch containment assembly 126. Such an engagement between the first housing 136 and the second housing 138 ensures a relative ease in assembly and disassembly of the first housing 136 relative to the second housing 138. Both the housings 136 and 138 may be made from considerably lightweight, chemically stable and lubricant resistive materials, and may include high-grade plastics, polymers, and the like.

The first housing 136 includes a first passage 140, which is structured co-axially relative to the first housing 136. The first passage 140 is fluidly connected with an inlet connector 146. The inlet connector 146 is arranged and extended outwardly to the first housing 136. In so doing, it is possible for the inlet connector 146 to be connected to the first connector 132 of the inlet tube portion 128. To complement such a connection, the inlet connector 146 is configured as a male connector. A connection between the first connector 132 and the inlet connector 146 may be similar to the connection between the first connector 132 and the second connector 134. By way of this arrangement, fluid communication between the inlet tube portion 128 and the first passage 140 of the first housing 136 is established.

Similarly, the second housing 138 includes a second passage 142, which is structured co-axially relative to the second housing 138. The second passage 142 is fluidly connected with an outlet connector 148. The outlet connector 148 is arranged and extended outwardly to the second housing 138. In this manner, it is possible for the outlet connector 148 to be connected to the second connector 134 of the outlet tube portion 130. To complement such a connection, the outlet connector 148 is a female connector. A connection between the second connector 134 and the outlet connector 148 may be similar to the connection between the first connector 132 and the second connector 134. By way of this arrangement, fluid communication between the outlet tube portion 130 and the second passage 142 of the second housing 138 is established. In a normal working mode, the first housing 136 remains connected with the second housing 138.

The second passage 142 includes a step portion 150, which is generally a concentrically structured straight-cut recess defined relative to the second passage 142. The step portion 150 is formed so as to define a co-axially disposed interface between the first passage 140 and the second passage 142. The step portion 150 includes a larger diametrical gap than that of the first passage 140 and the second passage 142. The step portion 150 may be optionally structured in the first housing 136. Alternatively, a portion of both the first passage 140 and the second passage 142 may define the step portion 150.

The step portion 150 provides accommodation to the patch member 152 within the patch containment assembly 126. As the step portion 150 is formed in the second passage 142, a containment of the patch member 152 is envisioned within the second housing 138. However, if the step portion 150 is structured in the first passage 140 an accommodation of the patch member 152 may be contemplated in the first housing 136 as well. Moreover, as configurations are envisioned where the step portion 150 extends into both the passages 140 and 142, it is possible for the patch member 152 to be accommodated partially in both the second housing 138 and the first housing 136. In yet other embodiments, a conically shaped step portion 150 is also possible.

The patch member 152 includes outer contours and peripheral configurations that facilitates a suitably accommodation within the step portion 150. The patch member 152 may be a conventionally available sieve membrane or a screen unit, which facilitates capture of contaminants and debris flowing in the lubricant that passes through and across the bypass line 122. The patch member 152 generally physically separates the first passage 140 from the second passage 142, but retains fluid communicability between the first passage 140 and the second passage 142.

Referring to FIG. 3, upon an assembly between the first housing 136 and the second housing 138, the passages 140 and 142 are aligned co-axially with each other. This arrangement of the passages 140 and 142 defines a containment passage 144, which acts as a through channel, defined along a height or an elongation of the patch containment assembly 126. With the connection of the inlet connector 146 to the first connector 132, and the outlet connector 148 to the second connector 134, the containment passage 144 forms a supplementary and an extended portion of the bypass line 122. In so doing, a lubricant flowing through the bypass line 122, flows through the containment passage 144 of the patch containment assembly 126, as well.

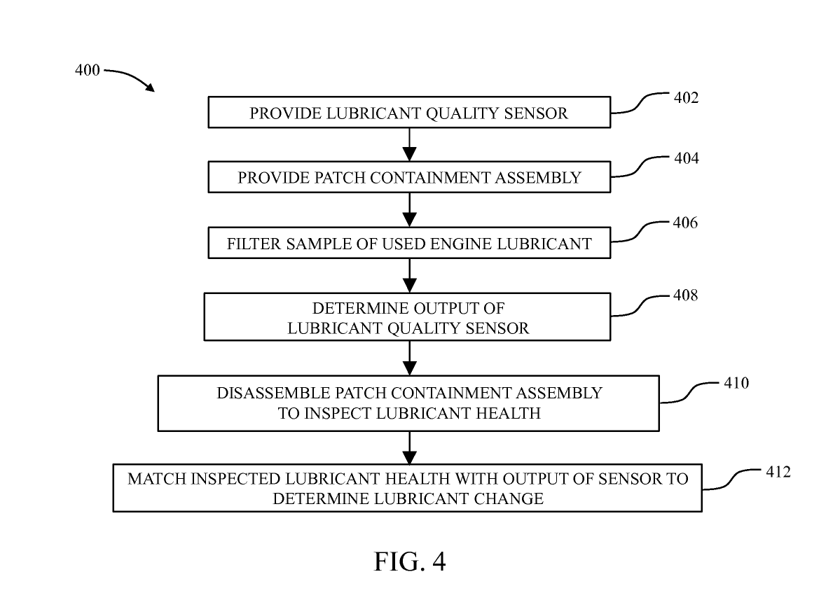

Referring to FIG. 4, an exemplary method of operation of the health analysis system 118 is described by means of a flowchart 400. The method is discussed in conjunction with FIGS. 1, 2 and 3. The method begins at step 402.

At step 402, an operator provides the lubricant quality sensor as part of the health analysis system 118. The provision of the lubricant quality sensor 120 may be envisioned as being the application of an existing sensor located within the lubrication system 104, which is able to determine at least one quality parameter of the lubricant. The method proceeds to step 404.

At step 404, the operator provides the patch containment assembly 126. This stage involves the installation of the patch containment assembly 126 into the bypass line 122 of the lubrication system 104. This installation includes a connection of the first connector 132 with the inlet connector 146 of the first housing 136, and a connection of the second connector 134 with the outlet connector 148 of the second housing 138. The method proceeds to step 406.

At step 406, a sample lubricant from the lubrication line 112 is made to pass through the bypass line 122. In so doing, at least a portion of the sample lubricant flows through the containment passage 144 and across the patch member 152. The method proceeds to step 408.

At step 408, the operator determines an output of the lubricant quality sensor 120. This output is recorded. The method proceeds to step 410.

At step 410, the operator disassembles the patch containment assembly 126, which involves a quick disengagement of the first housing 136 from the second housing 138. A visual inspection of the patch member 152 ascertains a lubricant health and a health of the lubrication system 104. This is determined as as an output of the patch containment assembly 126. The method proceeds to end step 412.

At end step 412, the operator matches the output of the patch containment assembly 126 and the lubricant quality sensor 120, based on which a requirement for a change of the lubricant is proposed.

### INDUSTRIAL APPLICABILITY

In operation, an operator connects the patch containment assembly 126 to the bypass line 122. This connection is facilitated by a connection between the first connector 132 of the inlet tube portion 128 and the inlet connector 146 of the first housing 136, and a connection between the second connector 134 of the outlet tube portion 130 and the outlet connector 148 of the second housing 138. Having established this connection, the operator activates the lubrication system 104 and allows a sample lubricant to flow through the bypass line 122. Since the bypass line 122 is installed with the patch containment assembly 126, sample lubricant flows through the containment passage 144 and is forced through the patch member 152. As a flow of the sample lubricant progresses, the patch member 152 captures containments within the flowing sample lubricant.

During a determination of the quality of lubrication and the health of the lubrication system 104, the operator disassembles the patch containment assembly 126. This disassembly may solely incur the removal of the first housing 136 from the second housing 138, while a connection between the inlet tube portion 128 and the first housing 136 and a connection between the outlet tube portion 130 and the second housing 138, remains unaltered. This disassembly between the first housing 136 and the second housing 138 allows the operator to analyze the containments captured by the patch member 152, visually. Thereafter, the operator checks the output from the lubricant quality sensor 120. If the parameter determined by the lubricant quality sensor 120 breaches a predefined threshold, and a containment entrapment within the patch member 152 exceeds a visually conceivable ordinal threshold, the operator raises a request for the change of the lubricant. However, if the patch member 152 is found to be relatively free of contaminants, the first housing 136 may be re-assembled to the second housing 138, and the lubrication system 104 may return to a normal working mode. In this manner, the lubricant of the lubrication system 104 is effectively monitored, and with the assistance of the health analysis system 118 it becomes more precise to determine an appropriate time for a lubricant change.

During normal working conditions, a failure in the lubrication system 104 leads to process down time, repairs, component procurement, material scrap, inconvenience, and the burden of cost and effort. In the absence of an effective diagnosis strategy, such as the health analysis system 118, an undetected minor technical glitch on the onset could result in major engine faults. In one example, problems associated with the bearing systems arising out of a lubrication fault may not become apparent until substantial damage has actually occurred. Thus, it is possible with the use of the health analysis system 118 to diagnose faults associated with the lubrication system 104 early so as to take a preventive action.

The patch containment assembly 126 is optionally removable from the lubrication system 104. In such a case, the bypass line 122 may simply act as an auxiliary passage that provides for a flow of a sample lubricant into the sump 114.

Advantageously, the portability of the patch containment assembly 126 and provision of standardized connectors 132, 134, 146, and 148, allows the patch containment assembly 126 to be applicable on various other ports of the lubrication system 104. This is because the connectors 132, 134, 146, and 148, allow for relatively easy installation and removal of the patch containment assembly 126 from the bypass line 122 of the lubrication system 104. Therefore, the patch containment assembly 126 may be applied to lubrication systems in machines and systems other than the engine 102. Moreover, lubrication systems applied in other pressure-lubricated components may contemplate the incorporation and use of the patch containment assembly 126. Moreover, it remains pertinent to perform such an analysis without the need to have external power. As the patch containment assembly 126 embodies a simplistic design and configuration, and with the usage of a limited a number of components, it makes the analysis free from complexity, external powering agents, and excess bulk. Moreover, such an assembly imparts freedom and modularity to the operation of analyzing the health of the lubrication system 104. To this end, the relatively simple procedure that involves the quick-twist disassembly of the first housing 136 from the second housing 138, and a reverse twist action to re-assemble the first housing 136 to the second housing 138, facilitates ease in monitoring a quality of the lubricant.

It should be understood that the above description is intended for illustrative purposes only and is not intended to limit the scope of the present disclosure in any way. Thus, one skilled in the art will appreciate that other aspects of the disclosure may be obtained from a study of the drawings, the disclosure, and the appended claim.

What is claimed is:

1. A health analysis system for an engine lubrication system, the engine lubrication system including a sump, a lubricant quality sensor to detect quality pertaining at least one parameter of a lubricant, and a conduit, the health analysis system comprising:
a bypass line connected from the conduit to the sump to facilitate passage of a sample engine lubricant;
a patch containment assembly including:
a first housing defining a first passage;
a second housing removably connectable to the first housing, the second housing defining a second passage;
a patch member being removably disposed within at least one of the first housing or the second housing, wherein the first passage being in fluid communication with the second passage through the patch member.
